(19) United States
(12) Patent Application Publication
(10) Pub. No.: US 2016/0242677 A1
(43) Pub. Date: Aug. 25, 2016

(54) SLATTED-FRAME MEASUREMENT DEVICE

(71) Applicant: THOMAS LORENZ, WALLENHORST (DE)

(72) Inventor: THOMAS LORENZ, WALLENHORST (DE)

(21) Appl. No.: 15/145,834

(22) Filed: May 4, 2016

Related U.S. Application Data
(63) Continuation of application No. PCT/EP2014/058806, filed on May 4, 2016.

(30) Foreign Application Priority Data
Nov. 5, 2013 (DE) 202013104953.0

(51) Int. Cl. A61B 5/107

(52) U.S. Cl. CPC A61B 5/1072; A61B 5/1075

(57) ABSTRACT

A measurement device for determining the dimensions of a specific user in order to optimally configure a slatted bedframe for the user. The device has a plurality of cross members, each cross member measuring the height of a specific measuring point on the user, such as shoulders, waist, hips. The cross members are coupled to each other by a height-adjustment system that is based on the standard height proportions of the human body. Manually adjusting the height of one cross member, for example, the shoulder height, results in the hip and waist cross members automatically being adjusted to the appropriate heights. Pairs of calipers are provided on each cross member that are adjustable in width, for measuring the widths at the selected measuring points.

### BACKGROUND INFORMATION

1. Field of the Invention

The invention relates to a measurement device for a slatted bedframe. More particularly, the invention relates to a measurement device for assembling a slated bedframe that is adapted to the body size of a specific customer.

2. Discussion of the Prior Art

A slatted bedframe is a rectangular frame in which a plurality of bed slats are assembled, parallel to each other, along the length of the frame. Typically, a mattress is placed on the slats. The loading applied to the individual slats by the person lying on such a slatted frame varies, depending on the size and weight of the person. This is the case even when a mattress and possibly other layers are placed between the person and the slatted frame. These differences in loading along the length of the frame can be taken into account when assembling the frame by using flexing or spring slats that have different spring rates in certain areas of the frame. In this way, the slatted frame can be optimally configured to the body of a specific user or to a specific body type with regard to height and weight.

Bed shops can assemble slatted frames that are adapted to specific users. The process of measuring the user and assembling the selected slats takes time and involves multiple repetitive steps. The time involved can be reduced, if the initial assembly of the slatted frame is assembled with spring slats that are already adapted as closely as possible to the load curve that is expected to be applied over the length of the slatted frame. A close approximation of the actual load curve can be provided, based on the outer dimensions of the specific user. But even in this case, measurements have to be taken of the specific person to be sure that the bedframe is optimally configured to support the body size and weight of the person.

What is needed, therefore, is a measurement device that will provide quick and reliable measurements of a specific user of a slatted bedframe, to enable assembly of a slatted frame that is optimally configured and adapted to the specific user. What is further needed is a standardized method of measuring the dimensions of the user as precisely as possible.

### BRIEF SUMMARY OF THE INVENTION

It is an object of the invention to provide a measurement device for measuring and recording certain dimensions that define the outer form of a specific user, in the shortest time possible. Typically, the measuring points for the pertinent dimensions are the shoulder height, and the widths at the shoulders, waist, and hips. The measurement device according to the invention provides a means of automatically determining the heights of the various measuring points, once an initial measuring point has been defined, thereby simplifying the measurement process.

The measurement device comprises a pair of calipers mounted on two or more horizontal cross members that are coupled to each other, such that they are arranged at different heights, and an adjustment system for adjusting the heights of the cross members. The cross members are adjustable to the height and the calipers adjustable to the width of each particular measurement point. The number of cross members is determined by the number of measuring points that are to be measured, i.e., one cross member for each measuring point.

The pair of calipers are slidably mounted on the respective cross member, and extend outward orthogonally to the cross member. They are spaced apart, so as to provide space between them to accommodate the person to be measured, hereinafter referred to simply as the “user.” The calipers are movable along the cross member and can be moved up against the user to obtain an accurate width dimension for the particular measuring point.

As mentioned above, each cross member is height-adjustably mounted on the measurement device and a cross member is provided for each point of measurement. The cross members are coupled to each other, such that, adjusting one cross member manually automatically results in the other cross member or members being adjusted an appropriate distance. It is possible to couple the cross members so that, by setting the height of one measuring point, such as the shoulder height, the remaining cross members may be automatically set to the heights of the other measuring points, because the proportions of the human body are well known. Knowing these proportions, it is possible to determine, with sufficient accuracy for the intended purpose, the height of the hips and waist, for example, after the shoulder height of the user has been set. Eliminating the need to individually and independently set the heights of each cross member greatly simplifies and accelerates the process of measuring the user.

The adjustment system includes a set of gears that control the height adjustments of each cross member. Providing the individual gears with different gear ratios makes it possible to set the height of one cross member and then have the height of the other one or more cross members to be adjusted different amounts. So, for example, assuming two users of different heights are being measured, and the difference in the height of their shoulders is 20 cm, the difference in the height of their hips is not 20 cm, but rather, a smaller amount. The gearing in the measurement device is designed to reflect the typical proportions of the human body and will adjust the heights of the hips accordingly.

Given that, with regard to constructing a slatted bedframe, the relevant measuring points for defining the body size of the user are the shoulder height, waist, and hips, four cross members may be provided in the measurement device: three for the measuring points already mentioned: shoulder, waist, and hips, and a fourth one for measuring the height of the user at the neck. The cross members are named accordingly: shoulder cross member, waist cross member, hip cross member, and neck cross member. The shoulder cross member is used to determine the shoulder width of the user at its widest place, i.e., next to the two upper arms; the neck cross member is placed at the level of the neck and the two calipers measure the shoulder height of the user. The measuring device uses this shoulder height measurement as the reference value for determining the body size of the user, and hence, the heights of the other measurement points, i.e., the shoulder, waist, and hips.

As mentioned above, these four cross member may all be coupled with each other, so that when the height adjustment of the first cross member is set, the other three cross members are automatically adjusted accordingly and brought to a height that corresponds to the correct height for respective measuring points. In the measurement device according to the invention, the first cross member, the one that is set manually, is preferably the neck cross member and the heights of the shoulder, waist, and hip cross members are adjusted automatically when the height of the neck cross member is set.

The two calipers that are provided on one cross member may also be metrologically coupled with each other and with some means of automatically indicating the width measurement, so that moving these two calipers toward each other or away from each other results in the corresponding change being shown in a manner that is then simple to read off. Several suitable embodiments of a measurement display are known in the field and to the person of skill in the art. For example, an optical detection means with an electronic display may be used, whereby a laser beam is used to determine the distance and the distance measurement is shown in the electronic display. Alternatively, a purely mechanical means may be employed, i.e., without the use of electronic means. In this case, a gear rack may be used to couple the two calipers on a cross member, so that the movement of one of the two calipers automatically results in the movement of the second caliper. Appropriately, a linear scale is provided on the cross member, such that the current position of one of the calipers indicates the distance between the two calipers on the scale.

In a particularly simple and advantageous embodiment of the measurement device, a spring-loaded rolled measuring tape may be used to determine the distance between the two calipers. One end of the tape is connected to one caliper and the free end to the other caliper. A movement of the calipers toward each other automatically results in the spring-loaded measuring tape rolling up and a movement farther apart pulls the tape against the spring action. Advantageously, one of the two calipers may include a protective housing for storing the rolled measuring tape. A window is provided in the housing, allowing one to simply read the measured distance between the calipers that is shown in the window. A groove or channel may be provided in the cross member to guide and protect the expanse of the tape that stretches between the two calipers.

It may be advantageous to provide an automatic operation of the calipers on one of the cross members. For example, the calipers for the neck cross member may be constructed to automatically move toward each other or away from each other, depending on the body size of the user, whereas the calipers for measuring the hip, waist, or shoulder widths are adjusted manually and are adapted to the specific contour of the user. To achieve the automatic width adjustment of the neck calipers, the measurement device according to the invention has a vertically oriented guide that widens toward the top. The calipers are movably guided along the respective cross member horizontally, but are also guided vertically on the guide contour. When the cross member is moved up or down, the distance of the calipers to each other also changes, due to the fact that the guide contour widens toward the top. An upward movement of the cross member on the guide contour, thus, moves the calipers outward and conversely, because the guide contour narrows in the downward direction, a downward movement of the cross member results in the calipers being moved toward each other along the cross member. This embodiment of the measurement device with the guide contour means that it is not necessary to individually and manually adjust the calipers of all four of the cross members provided on the measurement device when adapting them to the user. Rather, coupling the calipers with the neck cross member and the guide contour accelerates the measurement process, because, for at least one of the cross members, the calipers are adjusted in width automatically, depending on the height to which set of calipers has been set. And it makes sense to couple the neck calipers with the guide contour, because it is the shoulder height that is the relevant measurement, and not the actual width of the user's neck. Hence, it is possible to define the guide contour so that the narrowest and widest settings for the neck calipers are within general dimensions for the width of the shoulders at the top, to ensure that the neck calipers come to rest on the top of the user's shoulders.

A robust and simple construction of the measurement device according to the invention uses a plurality of gears to couple the cross members. These gears are rotatably mounted on a common shaft and, thus, rotate with the same angular velocity when the measurement device is adjusted. The gears, however, have different diameters, i.e., different gear ratios, so that the chains that run on the gears are each moved vertically different distances, according to the different diameters of the respective gears. In other words, the different gear ratios result in a proportional adjustment of the dependent second and possibly additional cross members relative to the adjustment of the first cross member.

A chain tension may be provided, as a means to ensure that the chains are reliably guided on the gears. The chains do not run in a loop around their respective gears, but rather, they are moved back and forth within a certain adjustment range. Advantageously, then, the chain tensioner may simply be a turnbuckle that is provided in a section of the chain run that does not travel over one of the mentioned gears or a return gear that is provided at another location. As mentioned, the gears have slightly different diameters and, because of this, the chain tensioner makes it possible to optimally adjust the chain to the desired length to ensure reliable functioning, independently of the chain pitch.

Advantageously a gear rack may be affixed to a chain. In this case, too, as with the mentioned turnbuckle, the chain does travel in a complete loop around the respective gear and a return gear that may also be provided, but rather, is moved a certain distance up and down when the height of the cross members is adjusted. For this reason, it isn't a problem to provide the gear rack on one of the chains. This gear rack meshes with a gear that can be set in rotational motion by means of a motor or by a handwheel or similar means and thereby achieve a height-adjustment of the cross members.

Advantageously operation of the adjustment system for cross members is done on the rear side of the measurement device. The front side of the measurement device is the side of the device that faces the user, and the opposite side is the rear side. Placing the actuation element for the adjustment system on the rear side of the device ensures that the measurement device is operated in the safest and most accident-free manner, excluding any possibility of collision of the operator with the moving cross members.

In a particularly simple and robust embodiment of the measurement device, the actuation element for adjusting the height of the cross members may be constructed as a handwheel. In this way, it is not necessary to connect an electrical power source to the measurement device, but rather, the measurement device is operated exclusively by mechanical means.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is a front plan view of the measurement device according to the invention, including a schematic contour of a user.

FIG. 2 is a side plan view of the measurement device of FIG. 1, showing the calipers extending from the front side of the device and an actuation means on the rear side of the device, and also showing that the contour is a flat silhouette.

FIG. 3 is a perspective view of the measurement device of FIGS. 1 and 2, showing the cross members adjusted to the heights of certain measurement points and the calipers measuring the width of the user at those measurement points.

FIG. 4 is a perspective rear diagonal view, showing the calipers mounted on the cross members.

FIG. 5 is a perspective side diagonal view, illustrating a linear scale.

FIG. 6 is a perspective top and front diagonal view, showing measuring tape housings incorporated into the caliper slide blocks.

FIG. 7 is a vertical cut through the upper area of the measurement device, showing the gear system for automatically adjusting the heights of the cross members.

FIG. 8 is a vertical cut through the area of the actuation element, showing the handwheel and the gear rack.

FIG. 9 is a vertical cut through the lower area of the measurement device, showing the chains running around the return gear.

FIG. 10 is a vertical cut through the middle area of the measurement device, showing the carrier plates and turnbuckles on a chain.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

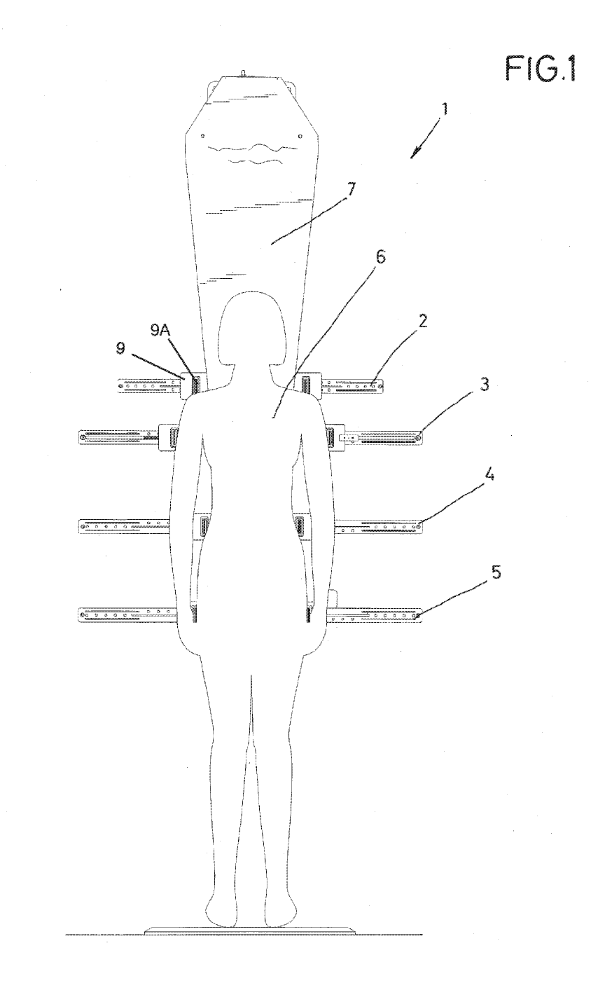

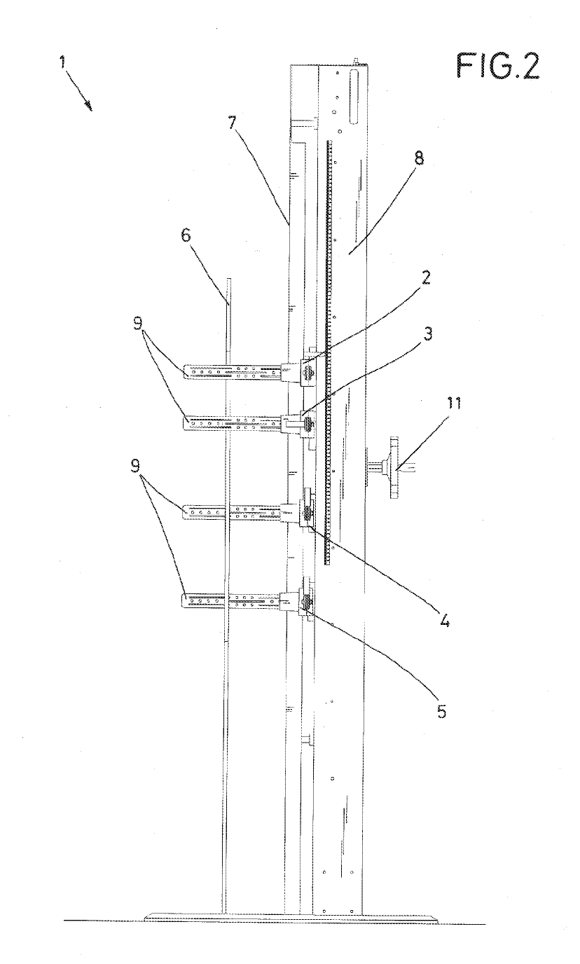

FIGS. 1-3 illustrate a measurement device 1 according to the invention for measuring the contour of a user, in order to assemble a slatted bedframe that is adapted to the body size of a specific user. The measurement device 1 comprises a plurality of cross members 2, 3, 4, 5, each cross member having a pair of calipers 9 that are slidably mounted on the respective cross members by means of a caliper block 9A and that extend horizontally and orthogonal to the cross members, creating a space for accommodating the user. A silhouette 6 of a user is shown in FIGS. 1 and 2, to illustrate how the cross members 2, 3, 4, 5 are set at certain measuring points on the user and how the calipers 9 are slidably movable along the cross members until they are up against the body of the user.

The defined measurement points for the intended purpose of the measurement device 1 are the neck, shoulder, waist, and hips of the user 6. The measurement device is constructed to rapidly and accurately determine the height and width of these measuring points. To do this, the cross members are set at the height of each measurement point and the calipers 9 on the respective cross members 2, 3, 4, 5 are adjusted to measure the width at that point.

In the embodiment shown, the plurality of cross members include a neck cross member 2, a shoulder cross member 3, a waist cross member 4, and a hip cross member 5. The neck cross member 2 is set at the height of the user's neck, thereby defining the shoulder height of the user. The shoulder cross member 3 is then set at the height of the shoulder in the area of the upper arm, the waist cross member 4 at the height of the waist, and the hip cross member 5 the height of the hips. It can be seen, that a vertically oriented guide contour 7 that widens toward the top is set up behind the silhouette 6.

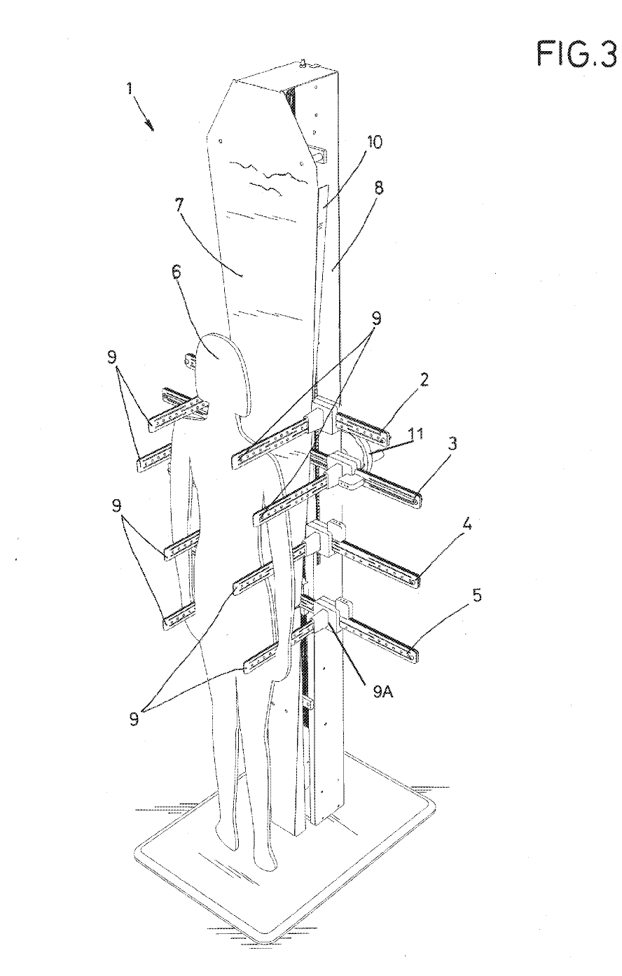

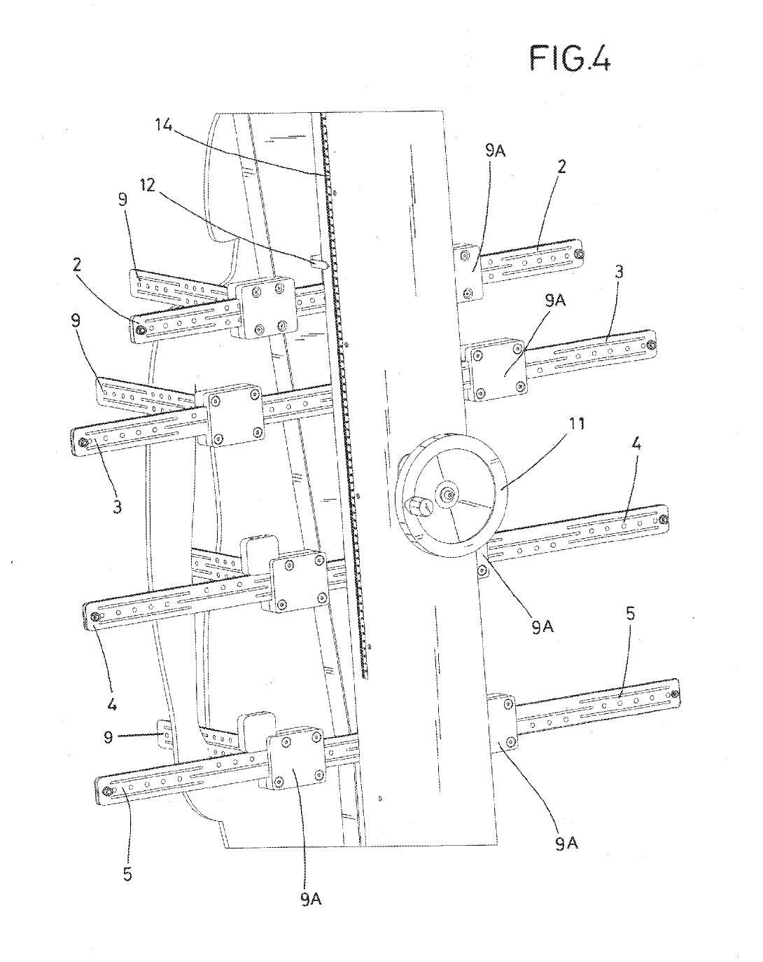

FIG. 3 shows that the guide contour 7 is constructed basically as a flat panel that gets wider toward the top and that has two side edges that are angled toward the back. These two side edges form an edge web 10 that extends toward the back on each side of the guide contour 7. This edge web 10 is captured in a groove 9B that is provided in the block 9A on the caliper 9 of the neck cross member 2, best seen in FIG. 4. This construction is symmetrical for both calipers 9 of the neck cross member 2, so that, when adjusting the height of the neck cross member 2, the width of both calipers 9 of this cross member is automatically pressed by the diagonally running edge web 10 either outward, namely, when the neck cross member 2 is moved upward, or together, namely, when the cross member 2 is moved downward.

The movement of the cross members 2, 3, 4, and 5 is done with the help of an actuation element 11 that is provided on the rear side of the measurement device 1 and is accordingly actuated from the rear side of the measurement device 1. In the embodiment shown, the actuation element 11 is constructed as a handwheel.

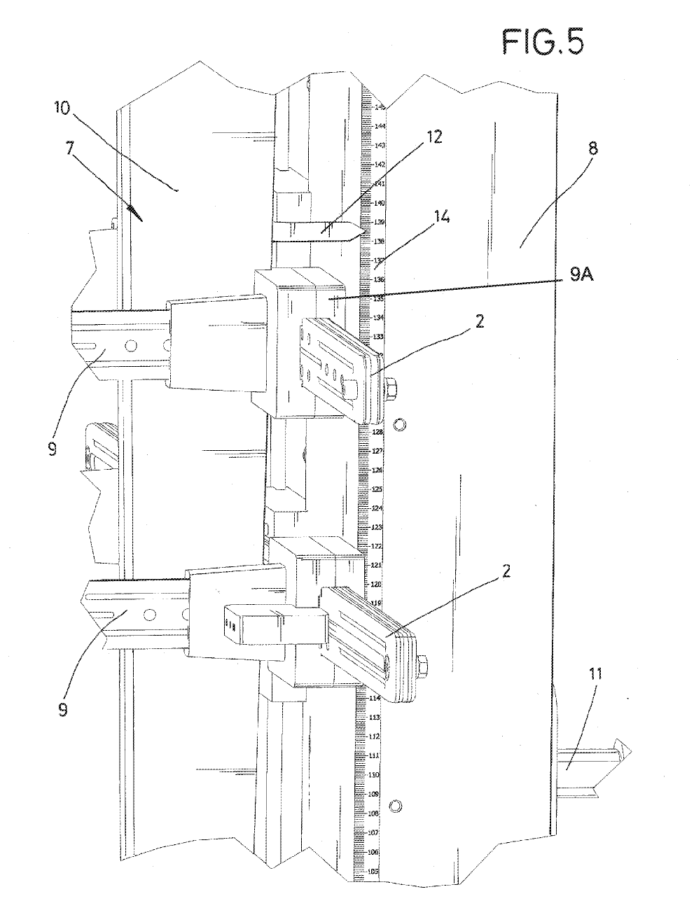

FIGS. 4 and 5 illustrate a linear scale 14 and a pointer 12. The scale 14 is provided along a side of the measurement device 1 and the pointer coupled to the neck cross member 2, such that it that moves up or down together with the neck cross member 2. In the embodiment shown, the pointer indicates the shoulder height of the user.

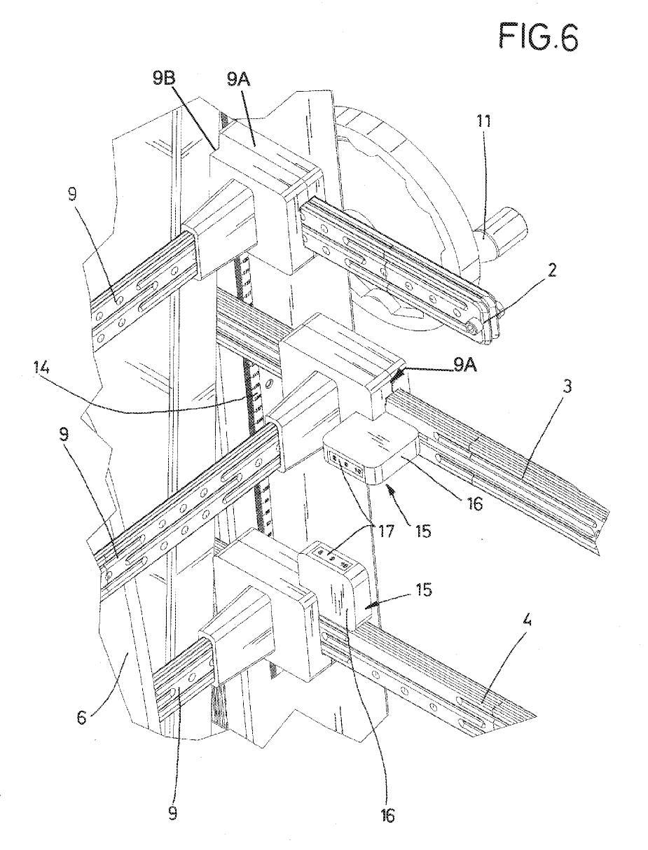

FIG. 6 illustrates details of the caliper block 9A and how the caliper 9 is mounted on the block 9A. The block 9A is slidably mounted on the respective cross member 2, 3, 4, 5 and the width of the calipers 9 on the respective cross member is set by sliding the block 9A along the cross member until the calipers are placed up against the user's body.

FIG. 6 also shows that a measuring tape 15 is provided on the caliper blocks 9A of the shoulder cross member 3 and the waist cross member 4. The measuring tape 15 is encased in a housing 16. Each of the housings 16 has a window 17. The free end of the measuring tape extends out beyond the housing 16 and is fastened to the respective second caliper 9 on the same cross member. A change in the distance of the two calipers 9 on the respective cross member 2, 3, 4, 5 results in the tape being pulled from or being rolled up in the housing 16. The measured distance between the two calipers 9 is then readable through the window 17.

The measurement device 1 according to the invention simplifies and reduces the amount of time required for the process of taking the body measurements of a specific user by providing automatic adjustment of one or more of the cross members 2, 3, 4, 5, based on the height that a first cross member is set at. In the embodiment described herein, the “first” cross member is represented by the neck cross member 2, although it is understood that a different measuring point other than the shoulder height may be used as the first cross member. In the embodiment shown, by manually setting the height of the neck cross member 2, the measurement device 1 automatically sets the height of the remaining shoulder, waist, and hip cross members 3, 4, and 5. An adjustment system that includes a plurality of gears 19, a corresponding plurality of chains 20, and an actuation element 11 is used to achieve this. The gears 19 and chains 20 are encased in a chain box 9, and the actuation element 11 is mounted on the rear face of the measurement device 1.

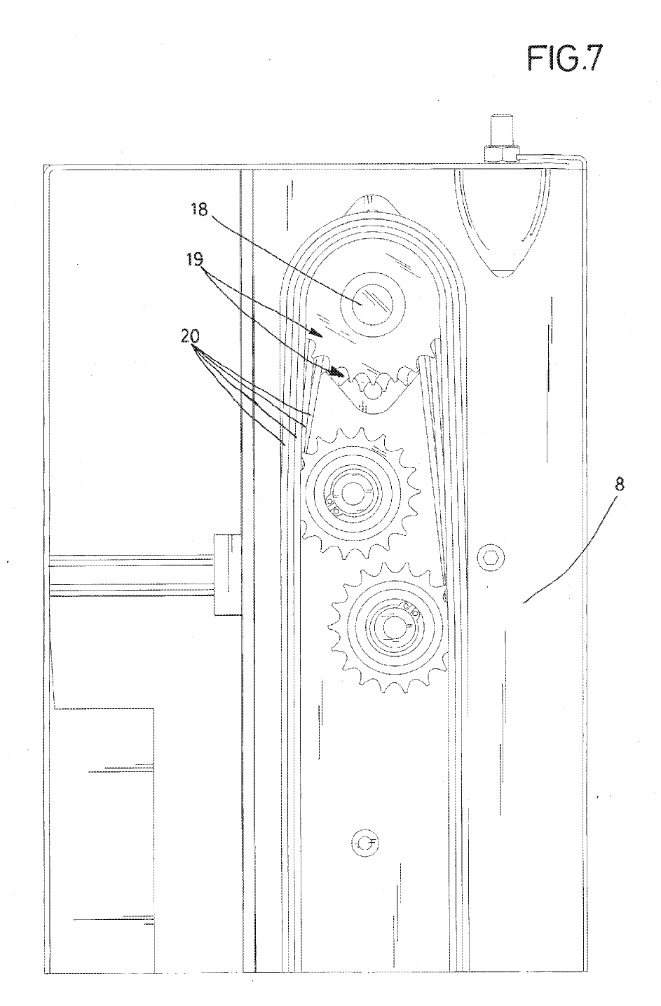

FIG. 7 shows a view into the upper area of the chain box 8, where a plurality of gears 19 are rotatably arranged on a common shaft 18. Gears with smaller diameters are provided on the shaft between the two gears that are visible in FIG. 7, and, because of the smaller diameter, the gear teeth of these gears are not visible. A gear 19 is provided for each cross member, hence, in this embodiment, a total of four gears 19, each gear having a different diameter, are provided, whereby each of these gears 19 is used to control the movement of a respective one of the four cross members 2, 3, 4, 5, i.e., the neck, shoulder, waist, or hip cross members, respectively. Four chains 20 are provided, one for each gear 19.

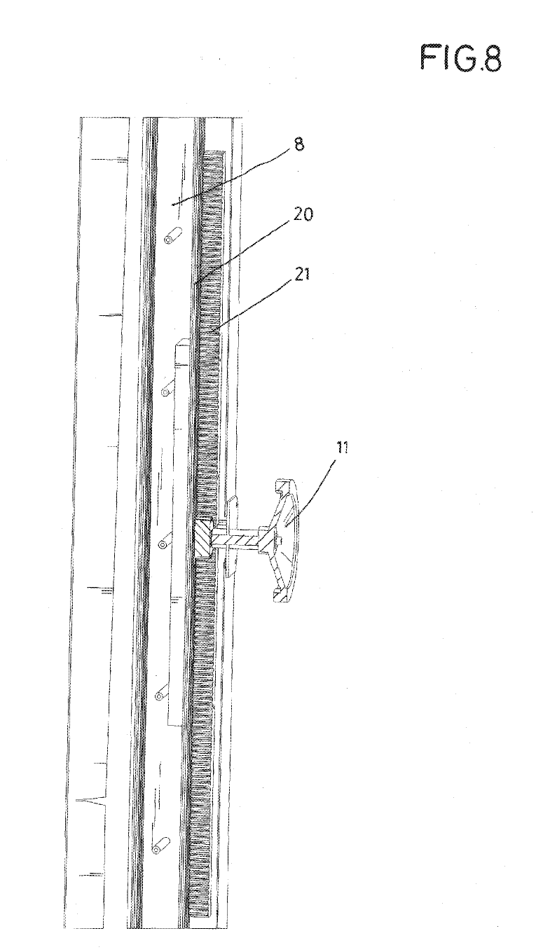

FIG. 8 is a view in the chain box 8 at a middle height, showing a gear rack 21 affixed to one of the four chains 20. The actuation element 11 operates on this gear rack 21. To this end, the actuation element 11 has a gear that is not visible in FIG. 8. This gear meshes with the rack 21. Turning the handwheel 11 moves the rack 21 up or down and, thus, also moves the chain 20 that is fixedly connected to this rack 21. On this manner, the gear 19 that cooperates with this chain 20 is rotated. The other gears 19 that are also mounted on the same shaft 18 are also rotated about the same angle of rotation. Due to the different diameters of the gears 19, the four chains 20 are moved a different distance upward or downward and, as a result, the cross members 2, 3, 4, 5 that are connected with these chains 19 are raised or lowered different distances.

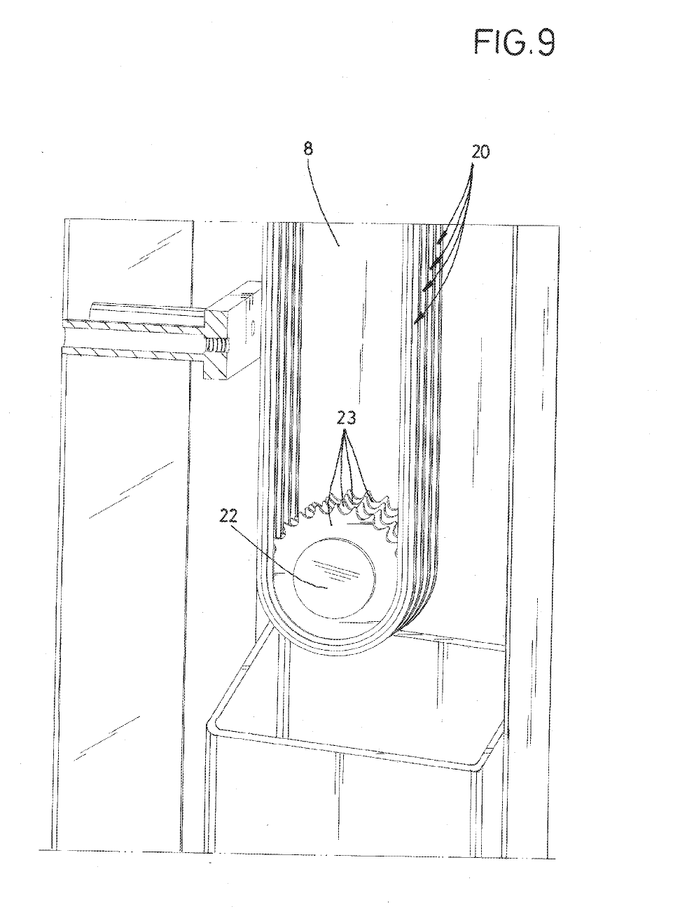

FIG. 9 shows a view into the lower area of the chain box 8, illustrating a return shaft 22, on which four return gears 23 are mounted so as to rotate freely, one return gear 23 for each gear 19, so that allocated to each cross member 2, 3, 4, 5 is a corresponding return gear 23 and chain 20.

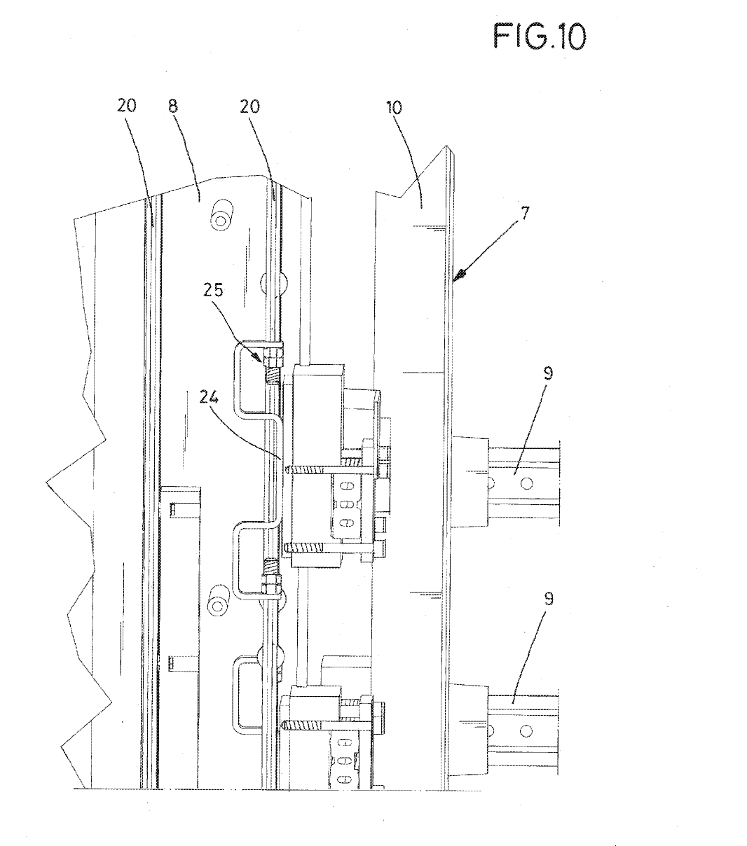

FIG. 10 is a view inside the chain box 8. One can see that the chains 20 have carrier plates 24 and that each of the cross members 2, 3, 4, 5 is connected to a respective one of the carrier plates 24, so that, depending on the movement of the chain 20, the cross members 2, 3, 4, 5 are moved up or down. Further, FIG. 10 illustrates turnbuckles 25 on the chains 20. These turnbuckles 25 serve to adjust the tension on the chain 20 and also to optimally adapt the length of the chain to the circumference or diameter of the respective gear 19.

It is understood that the embodiments described herein are merely illustrative of the present invention. Variations in the construction of the measurement device according to the invention may be contemplated by one skilled in the art without limiting the intended scope of the invention herein disclosed and as defined by the following claims.

What is claimed is:

1. A measurement device for determining the dimensions of a specific user at several measuring points on the user's body, the measurement device comprising:
a plurality of cross members that include at least a first cross member and a second cross member, each cross member of the plurality of cross members being horizontally oriented and allocated to a specific one of the measuring points;
a pair of calipers slidably mounted on a respective one of each cross member, the calipers extending horizontally and orthogonally outward from the respective cross member and being adjustable to a width of the specific one of the measuring points; and
an adjustment system for adjusting a height of each of the cross members to a height of a respective one of the several measuring points on the user's body, the adjustment system including a plurality of gears corresponding in number to the plurality of cross members, the plurality of gears operating at a common angle of rotation;
wherein each cross member is coupled to a specific one of the gears; and
wherein a height adjustment of the first cross member automatically results in a height adjustment of the second cross member, the height adjustment of the first cross member being a first distance and the height adjustment of the second cross member being a second distance that is different from the first distance.

2. The measurement device of claim 1,
wherein the plurality of cross members includes four cross members: a neck cross member, a shoulder cross member, a waist cross member, and a hip cross member;
wherein the calipers on the neck cross member are positionable at both sides of the user's neck, the calipers on the shoulder cross member positionable at both sides of the user's shoulders, the calipers on the waist cross member positionable at both sides of the user's waist, and the calipers of the hip cross member positionable at both sides of the user's hips; and
wherein a height adjustment of one of the four cross members automatically results in a height adjustment of the remaining three cross members, the height adjustment of each of the four cross members being a different amount.

3. The measurement device of claim 1, further including a display on one of the calipers of the pair of calipers;
wherein the pair of calipers are metrologically coupled with each other, such that a distance between the pair of calipers is shown in the display.

4. The measurement device of claim 3, the pair of calipers further including a spring-loaded rolled measuring tape that is mounted on one caliper of the pair of calipers, and wherein a free end of the measuring tape is connected to the other caliper.

5. The measurement device of claim 4, wherein the measuring tape is stored in a housing and wherein the housing has a window that serves as the display.

6. The measurement device of claim 1 further comprising a guide contour that is vertically oriented and that has a width dimension that widens toward a top of the guide contour;
wherein the pair of calipers on the first cross member is coupled with the guide contour such, that moving the first cross member upward automatically results in an increase in distance and moving downward automatically results in a decrease in distance between the pair of calipers.

7. The measurement device of claim 1, the cross member adjustment system including a plurality of chains corresponding in number to the plurality of gears;
wherein the plurality of gears are rotatably mounted on a common shaft, each gear of the plurality of gears having a different gear ratio; and
wherein each one of the plurality of chains runs along a specific one of the gears, and wherein each chain is connected to a specific one of the plurality of cross members.

8. The measurement device of claim 7, the cross member adjustment system including a turnbuckle on each chain for enabling a length adjustment of the chain.

9. The measurement device of claim 7, the cross member adjustment system further including a gear rack that is affixed to a specific one of the chains, the gear rack meshing with one of the gears that is rotatably movable for adjusting a height of the cross members.

10. The measurement device of claim 1, wherein the measurement device has a front side that faces the user and an opposite side that is a rear side; and
wherein an actuation element is provided on the rear side, by means of which the first cross member is selectively moved upward or downward.

11. The measurement device of claim 10, wherein the actuation element is constructed as a handwheel.
